SEQUENCE LISTING (8 sequences)

SEQ ID NO: 1 - DNA, 2256 bp, Unknown
  Synthetic Construct
atgaatcggttcatgaagaagctattctctatgttcttggcgctagccctgatcgttggctatacggcggcctatcctctgcccgcagacgccgcggcgtccggacaatcattagggccggtaacatcgaaggatgtcatttatcagattttgacggatcgcttctatgacggagatcccgcgaacaacattcccccaggcacacccccggagctgttcaacgatgacaacggggatggacgcggcgacggaaccgatctgaacaaatatcaaggcggcgattggaaaggcatccaggagaaaatcccgtacctgaaaaatatgggcattacggcggtatggatctccgctccgtacgagaaccgggagaacctgatcgccggcatgtacgcatcctatcatggctatcatgcgcggaattacttcgcaaccaaccctcatttcgggaaaatgcaggacttcacggcgctggtcgatgcgctccatgacaacgggatcaaggtggttattgatttcgtgaccaaccattccggtccgcggccggacggcgacggcgtgctgtatgaaccagatcgcgacagctccggccagtatgtgttcgacccggacggcaacccaatcgattacaacggcgacggcaaggtcgagaaccgaatcgccgacatcctcaacgatacgaacggctttttccatcatgaagggaaccggccggacagcgatacatccaaattcggataccggcataaggaattggcctcgctcgccgactattcccaggagaacggcgtcgtcatcgaacatttggagaaggcgggcaagttctggaaggcgaagggcattgacggttttcgccatgatgccacgctgcacatgaacccggcctttgtgaaaggattcaaggatgcgatcgattctgccccaggccgtccggtgacccacttcggggaatttttcattggccggcctgatccgaagtatgacgaataccgaacgttccctgatcggacaggcgtcaacaatctggacttcgaatattacaatgcgaaccggcaagcgtttggtgacttcagcagaagcatgagcgatttcggacaaatgctcgtacagacgagcgcggactatacggtagaaaatcaggccgtcacctttatcgacaatcatgacgtgtcgcgcttccgttatatacagccgaatgacaagccctatcatgcttcgcttgccgtgcttcttacctcgcgcggaatcccgaacctttattatggcacggagcaatacttgaatccgggccatggcgggtcggatgcaggccgcttgttcctgcaggcggcggccccggcgttcagcgagcagaccgtagcctaccgccttatcggcaagctgtcggccctgcggcagtccaatgacgccttggcatacgggacgaccgatattttgttcagcaatgacgatgcgctcgtgtacaagcgccagttcttcgacaagcaggtgattgtcgccgtgaaccggcagccggatcgttccgtatccgtaccggccttgacgacgacccttccggcgggaagctataccgacgcattggacggtttgctgtacggacggacaatgaccgtcgttgatcagaacgcagagcggcagattccagcctttacgctcgttggcggcgaagtcagcgtctggtcggataatccgccagccgatcctggggaaccgcttatcggcgatgtcgtctcaacgatggggcgtccagggaacaccgtatacatctatggcaccggactgagcggtggggctaccgttgcattcgggtctcagcaggctgcggtcgtatccgcacaggataaccggattgcggccgtggtgccgaacgtgcctgccggcgactacgcgattactgtcacgaaagacggaaagacgagcaacccgttccggtaccaggtgctgggcggggatcaagtgcaggtcattttccacgtcaataagacgacgcagccgggacagaatgtctatgtcgttggcgatattgccgaactgggggcctggaatccggacaatgtgctcgattcgttcatgaatccgaattatccggactggttcctgccggttagcgtgccagccggggcggcctttcagttcaaattcatcatcaaagacgcggcaggccatgtcacctgggagggcggggcgaaccgcatgtttaccgccacatcaaacccgacgggaacaagcgacactccggtgtacaactggcagccttag

SEQ ID NO: 2 - protein, 751 aa, Unknown
  Synthetic Construct
MNRFMKKLFSMFLALALIVGYTAAYPLPADAAASGQSLGPVTSKDVIYQILTDRFYDGDPANNIPPGTPPELFNDDNGDGRGDGTDLNKYQGGDWKGIQEKIPYLKNMGITAVWISAPYENRENLIAGMYASYHGYHARNYFATNPHFGKMQDFTALVDALHDNGIKVVIDFVTNHSGPRPDGDGVLYEPDRDSSGQYVFDPDGNPIDYNGDGKVENRIADILNDTNGFFHHEGNRPDSDTSKFGYRHKELASLADYSQENGVVIEHLEKAGKFWKAKGIDGFRHDATLHMNPAFVKGFKDAIDSAPGRPVTHFGEFFIGRPDPKYDEYRTFPDRTGVNNLDFEYYNANRQAFGDFSRSMSDFGQMLVQTSADYTVENQAVTFIDNHDVSRFRYIQPNDKPYHASLAVLLTSRGIPNLYYGTEQYLNPGHGGSDAGRLFLQAAAPAFSEQTVAYRLIGKLSALRQSNDALAYGTTDILFSNDDALVYKRQFFDKQVIVAVNRQPDRSVSVPALTTTLPAGSYTDALDGLLYGRTMTVVDQNAERQIPAFTLVGGEVSVWSDNPPADPGEPLIGDVVSTMGRPGNTVYIYGTGLSGGATVAFGSQQAAVVSAQDNRIAAVVPNVPAGDYAITVTKDGKTSNPFRYQVLGGDQVQVIFHVNKTTQPGQNVYVVGDIAELGAWNPDNVLDSFMNPNYPDWFLPVSVPAGAAFQFKFIIKDAAGHVTWEGGANRMFTATSNPTGTSDTPVYNWQP

SEQ ID NO: 3 - DNA, 20 bp, Artificial Sequence
  oligonucleotide
tcaaaattgttcctgaatgc

SEQ ID NO: 4 - DNA, 18 bp, Artificial Sequence
  oligonucleotide
gcaaggacaacaatgctc

SEQ ID NO: 5 - DNA, 19 bp, Artificial Sequence
  oligonucleotide
tgctgtatgaaccagatcg

SEQ ID NO: 6 - DNA, 20 bp, Artificial Sequence
  oligonucleotide
actggtacacgatgaaatcc

SEQ ID NO: 7 - DNA, 20 bp, Artificial Sequence
  oligonucleotide
tattttgttcagcaatgacg

SEQ ID NO: 8 - DNA, 19 bp, Artificial Sequence
  oligonucleotide
cgtgacagtaatcgcgtag

(19) United States
(12) Patent Application Publication
(10) Pub. No.: US 2010/0112637 A1
(43) Pub. Date: May 6, 2010

(54) Polypeptides Having Alpha-Amylase Activity and Polynucleotides Encoding Same

(75) Inventors: Martin Borchert, Copenhagen Oe (DK); Anders Viksoe-Nielsen, Slangerup (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/524,607

(22) PCT Filed: Jan. 31, 2008
(86) PCT No.: PCT/EP2008/051166

§ 371 Date: Aug. 3, 2009

(30) Foreign Application Priority Data
Feb. 1, 2007 (EP) 07101570.5

(51) Int. Cl. C12P 21/00; C12N 9/28; C12P 19/14; C07H 21/00; C12N 15/74; C12N 1/15

(52) U.S. Cl. 435/69.1; 435/202; 435/99; 536/23.2; 435/320.1; 435/254.11

(57) ABSTRACT

The present invention relates to isolated polypeptides having alpha-amylase activity and isolated polynucleotides encoding the polypeptides. The invention also relates to nucleic acid constructs, vectors, and host cells comprising the polynucleotides as well as methods for producing and using the polypeptides.

### REFERENCE TO SEQUENCE LISTING

This application contains a Sequence Listing in computer readable form. The computer readable form is incorporated herein by reference.

### FIELD OF THE INVENTION

The present invention relates to isolated polypeptides having alpha-amylase activity and isolated polynucleotides encoding the polypeptides. The invention also relates to nucleic acid constructs, vectors, and host cells comprising the polynucleotides as well as methods for producing and using the polypeptides.

### DESCRIPTION OF THE RELATED ART

Alpha-amylases (alpha-1,4-glucan-4-glucanohydrolases, EC. 3.2.1.1) constitute a group of enzymes which catalyze hydrolysis of starch and other linear and branched 1,4-glucosidic oligo- and polysaccharides.

For a number of years alpha-amylase enzymes have been used for a variety of different purposes, the most important of which are starch liquefaction, textile desizing, starch modification in the paper and pulp industry, and for brewing, ethanol production and baking.

There is a very extensive body of patent and scientific literature relating to this industrially very important class of enzymes. A number of alpha-amylases referred to as “Termamyl®-like alpha-amylases” and variants thereof are known from, e.g., WO 90/11352, WO 95/10603, WO 95/26397, WO 96/23873 and WO 96/23874. Termamyl®-like alpha-amylases are very thermostable and therefore suitable for processes carried out at high temperatures such as starch liquefaction in dextrose production processes.

Another group of alpha-amylases are referred to as “Fungamyl™-like alpha-amylases”, which are alpha-amylases related or homologous to the alpha-amylase derived from Aspergillus oryzae. The Fungamyl-like alpha-amylases have a relatively low thermostability; the commercial product sold under the trade name FUNGAMYL™ by Novozymes A/S, Denmark, has an optimum around 55° C., and is not suitable for processes carried out at high temperatures. Fungamyl™-like alpha-amylases are today used for making syrups for, e.g., the brewing industry.

Clearly, it would be advantageous to provide alternative alpha-amylases having different properties than the previously known alpha-amylases, in particular alpha-amylases having high activity at a neutral or acidic pH.

It is an object of the present invention to provide polypeptides having alpha-amylase activity and polynucleotides encoding the polypeptides.

### SUMMARY OF THE INVENTION

The present invention provides isolated polypeptides having alpha-amylase activity or starch binding activity, selected from the group consisting of:

(a) a polypeptide having an amino acid sequence which has at least 60% identity with amino acids 1 to 719 of SEQ ID NO: 2;

(b) a polypeptide which is encoded by a nucleotide sequence which hybridizes under at least medium stringency conditions with (i) nucleotides 1 to 2256 of SEQ ID NO: 1, or (ii) a complementary strand of (i); and

(c) a polypeptide having an amino acid sequence derived from amino acids 1 to 719 of SEQ ID NO: 2 by substitution (particularly conservative substitution), deletion, and/or insertion of one or more amino acids.

The present invention also relates to isolated polynucleotides encoding polypeptides having alpha-amylase activity, selected from the group consisting of:

(a) a polynucleotide having at least 60% identity with nucleotides 97 to 2256 of SEQ ID NO: 1; and

(c) a polynucleotide which hybridizes under medium stringency conditions with (i) nucleotides 97 to 2256 of SEQ ID NO: 1, or (ii) a complementary strand of (i).

The present invention also relates to nucleic acid constructs, recombinant expression vectors, and recombinant host cells comprising the polynucleotides.

The present invention further relates to methods for producing such polypeptides having alpha-amylase activity comprising (a) cultivating a recombinant host cell comprising a nucleic acid construct comprising a polynucleotide encoding the polypeptide under conditions conducive for production of the polypeptide; and (b) recovering the polypeptide.

The present invention further relates to nucleic acid constructs comprising a gene encoding a protein, wherein the gene is operably linked to a nucleotide sequence encoding a signal peptide consisting of nucleotides 1 to 96 of SEQ ID NO: 1, wherein the gene is foreign to the nucleotide sequence encoding the signal sequence.

The alpha-amylase (polypeptide with alpha-amylase activity) may be used in various processes for producing maltodextrins, comprising incubating starch or a starch hydrolysate with the alpha-amylase so as to hydrolyze alpha-1,4 bonds in the starch or starch hydrolysate.

Thus, the alpha-amylase may be used in the starch industry, the food processing industry, the textile industry, and the detergent industry, e.g. for starch liquefaction, saccharification of liquefied starch, textile desizing, starch modification in the paper and pulp industry, brewing, ethanol production and baking. The alpha-amylase may be used for producing an enzymatically modified starch derivative, wherein the alpha-amylase is used for liquefying and/or saccharifying starch; for producing syrups (e.g., high maltose syrups), wherein the alpha-amylase is used for liquefaction of starch and/or in the saccharification of liquefied starch; for desizing textile, wherein the alpha-amylase is used for treating the textile; and for brewing, wherein the alpha-amylase is added during fermentation of wort; for alcohol production, wherein the alpha-amylase is used for liquefaction of starch in a distillery mash; and in a process, wherein a dough product comprising the alpha-amylase is baked. It may be used in a starch conversion process for liquefaction and/or saccharification; for liquefying starch in a high maltose syrup production process; for textile desizing; for producing alcohol; for brewing; and for baking.

### DEFINITIONS

Alpha-amylase activity: The term “alpha-amylase activity” is defined herein as a 1,4-glucan-4-glucanohydrolases (EC. 3.2.1.1) activity which catalyzes the hydrolysis of starch and other linear and branched 1,4-glucosidic oligo- and polysaccharides. Alpha-amylase assays are described below.

Starch binding activity: The term “starch binding activity” is understood as the ability of a polypeptide to bind natural starch. For purposes of the present invention starch binding activity is understood as the binding activity of starch binding carbohydrate binding modules as reviewed by A. B. Boraston in (Boraston, A. B. et al. 2004. Carbohydrate-binding modules: fine-tuning polysaccharide recognition. Biochem. J. 382:769-781).

Isolated polypeptide: The term “isolated polypeptide” as used herein refers to a polypeptide which is at least 20% pure, preferably at least 40% pure, more preferably at least 60% pure, even more preferably at least 80% pure, most preferably at least 90% pure, and even most preferably at least 95% pure, as determined by SDS-PAGE.

Substantially pure polypeptide: The term “substantially pure polypeptide” denotes herein a polypeptide preparation which contains at most 10%, preferably at most 8%, more preferably at most 6%, more preferably at most 5%, more preferably at most 4%, at most 3%, even more preferably at most 2%, most preferably at most 1%, and even most preferably at most 0.5% by weight of other polypeptide material with which it is natively associated. It is, therefore, preferred that the substantially pure polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation.

The polypeptides of the present invention are preferably in a substantially pure form. In particular, it is preferred that the polypeptides are in “essentially pure form”, i.e., that the polypeptide preparation is essentially free of other polypeptide material with which it is natively associated. This can be accomplished, for example, by preparing the polypeptide by means of well-known recombinant methods or by classical purification methods.

Herein, the term “substantially pure polypeptide” is synonymous with the terms “isolated polypeptide” and “polypeptide in isolated form.”

Identity: The relatedness between two amino acid sequences is described by the parameter “identity”.

For purposes of the present invention, the alignment of two amino acid sequences is determined by using the Needle program from the EMBOSS package (http://emboss.org) version 2.8.0. The Needle program implements the global alignment algorithm described in Needleman, S. B. and Wunsch, C. D. (1970) J. Mol. Biol. 48, 443-453. The substitution matrix used is BLOSUM62, gap opening penalty is 10, and gap extension penalty is 0.5.

The degree of identity between a first and a second amino acid sequence is calculated as the number of exact matches in an alignment of the two sequences, divided by the length of the first or the second sequence, whichever is shorter. The result is expressed in percent identity.

An exact match occurs when the two sequences have identical amino acid residues in the same positions of the overlap. The length of a sequence is the number of amino acid residues in the sequence.

For purposes of the present invention, the degree of identity between two nucleotide sequences is determined according to the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

In a particular embodiment, the percentage of identity of an amino acid sequence of a polypeptide with, or to, amino acids 1 to 719 of SEQ ID NO: 2 is determined by i) aligning the two amino acid sequences using the Needle program, with the BLOSUM62 substitution matrix, a gap opening penalty of 10, and a gap extension penalty of 0.5; ii) counting the number of exact matches in the alignment; iii) dividing the number of exact matches by the length of the shortest of the two amino acid sequences, and iv) converting the result of the division of iii) into percentage. The percentage of identity to, or with, other sequences of the invention such as amino acids 1-719 of SEQ ID NO: 2 are calculated in an analogous way.

The polypeptides of the present invention have at least 20%, preferably at least 40%, more preferably at least 50%, more preferably at least 60%, more preferably at least 70%, more preferably at least 80%, even more preferably at least 90%, most preferably at least 95%, and even most preferably at least 100% of the alpha-amylase activity of the polypeptide consisting of the amino acid sequence shown as amino acids 1 to 719 of SEQ ID NO: 2.

Polypeptide Fragment: The term “polypeptide fragment” is defined herein as a polypeptide having one or more amino acids deleted from the amino and/or carboxyl terminus of SEQ ID NO: 2 or a homologous sequence thereof, wherein the fragment has biological activity, such as alpha-amylase activity or starch binding activity.

Allelic variant: The term “allelic variant” denotes herein any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Substantially pure polynucleotide: The term “substantially pure polynucleotide” as used herein refers to a polynucleotide preparation free of other extraneous or unwanted nucleotides and in a form suitable for use within genetically engineered protein production systems. Thus, a substantially pure polynucleotide contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polynucleotide material with which it is natively associated. A substantially pure polynucleotide may, however, include naturally occurring 5′ and 3′ untranslated regions, such as promoters and terminators. It is preferred that the substantially pure polynucleotide is at least 90% pure, preferably at least 92% pure, more preferably at least 94% pure, more preferably at least 95% pure, more preferably at least 96% pure, more preferably at least 97% pure, even more preferably at least 98% pure, most preferably at least 99%, and even most preferably at least 99.5% pure by weight. The polynucleotides of the present invention are preferably in a substantially pure form. In particular, it is preferred that the polynucleotides disclosed herein are in “essentially pure form”, i.e., that the polynucleotide preparation is essentially free of other polynucleotide material with which it is natively associated. Herein, the term “substantially pure polynucleotide” is synonymous with the terms “isolated polynucleotide” and “polynucleotide in isolated form.” The polynucleotides may be of genomic, cDNA, RNA, semisynthetic, synthetic origin, or any combinations thereof.

Nucleic acid construct: The term “nucleic acid construct” as used herein refers to a nucleic acid molecule, either single- or double-stranded, which is isolated from a naturally occurring gene or which is modified to contain segments of nucleic acids in a manner that would not otherwise exist in nature. The term nucleic acid construct is synonymous with the term “expression cassette” when the nucleic acid construct contains the control sequences required for expression of a coding sequence of the present invention.

Control sequence: The term “control sequences” is defined herein to include all components, which are necessary or advantageous for the expression of a polynucleotide encoding a polypeptide of the present invention. Each control sequence may be native or foreign to the nucleotide sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

Operably linked: The term “operably linked” denotes herein a configuration in which a control sequence is placed at an appropriate position relative to the coding sequence of the polynucleotide sequence such that the control sequence directs the expression of the coding sequence of a polypeptide.

Coding sequence: When used herein the term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG. The coding sequence may a DNA, cDNA, or recombinant nucleotide sequence.

Expression: The term “expression” includes any step involved in the production of the polypeptide including, but not limited to, transcription, post-transcriptional modification, translation, post-translational modification, and secretion.

Expression vector: The term “expression vector” is defined herein as a linear or circular DNA molecule that comprises a polynucleotide encoding a polypeptide of the invention, and which is operably linked to additional nucleotides that provide for its expression.

Host cell: The term “host cell”, as used herein, includes any cell type which is susceptible to transformation, transfection, transduction, and the like with a nucleic acid construct comprising a polynucleotide of the present invention.

Modification: The term “modification” means herein any chemical modification of the polypeptide consisting of the amino acids 1 to 719 of SEQ ID NO: 2 as well as genetic manipulation of the DNA encoding that polypeptide. The modification(s) can be substitution(s), deletion(s) and/or insertions(s) of the amino acid(s) as well as replacement(s) of amino acid side chain(s).

Artificial variant: When used herein, the term “artificial variant” means a polypeptide having alpha-amylase activity produced by an organism expressing a modified nucleotide sequence of SEQ ID NO: 1. The modified nucleotide sequence is obtained through human intervention by modification of the nucleotide sequence disclosed in SEQ ID NO: 1.

### DETAILED DESCRIPTION OF THE INVENTION

### Alpha-Amylases

In a first aspect, the present invention relates to isolated polypeptides having an amino acid sequence which has a degree of identity to the mature polypeptide of SEQ ID NO: 2 (i.e., amino acids 1 to 719) of at least 65%, at least 70%, at least 75%, at least 80%, at least 85%, at least 90%, at least 95%, or at least 97%, and which have alpha-amylase activity (hereinafter “homologous polypeptides”). In a preferred aspect, the homologous polypeptides have an amino acid sequence which differs by ten amino acids, preferably by five amino acids, more preferably by four amino acids, even more preferably by three amino acids, most preferably by two amino acids, and even most preferably by one amino acid from amino acids 1 to 719 of SEQ ID NO: 2.

A polypeptide of the present invention preferably comprises the amino acid sequence of SEQ ID NO: 2 or an allelic variant thereof; or a fragment thereof that has alpha-amylase activity or starch binding activity. In a preferred aspect, a polypeptide comprises the amino acid sequence of SEQ ID NO: 2. In another preferred aspect, a polypeptide comprises amino acids 1 to 719 of SEQ ID NO: 2, or an allelic variant thereof; or a fragment thereof that has alpha-amylase activity. In another preferred aspect, a polypeptide consists of the amino acid sequence of SEQ ID NO: 2 or an allelic variant thereof; or a fragment thereof that has alpha-amylase activity. In another preferred aspect, a polypeptide consists of the amino acid sequence of SEQ ID NO: 2.

In a second aspect, the present invention relates to isolated polypeptides having alpha-amylase activity which are encoded by polynucleotides which hybridize under very low stringency conditions, preferably low stringency conditions, more preferably medium stringency conditions, more preferably medium-high stringency conditions, even more preferably high stringency conditions, and most preferably very high stringency conditions with (i) nucleotides 1 to 2256 of SEQ ID NO: 1, (ii) a subsequence of (i), or (iii) a complementary strand of (i) or (ii) (J. Sambrook, E. F. Fritsch, and T. Maniatis, 1989, Molecular Cloning, A Laboratory Manual, 2d edition, Cold Spring Harbor, N.Y.). A subsequence of SEQ ID NO: 1 contains at least 100 contiguous nucleotides or preferably at least 200 contiguous nucleotides. Moreover, the subsequence may encode a polypeptide fragment which has alpha-amylase activity or starch binding activity.

The nucleotide sequence of SEQ ID NO: 1 or a subsequence thereof, as well as the amino acid sequence of SEQ ID NO: 2 or a fragment thereof may be used to design a nucleic acid probe to identify and clone DNA encoding polypeptides having alpha-amylase activity from various organisms according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. It is, however, preferred that the nucleic acid probe is at least 100 nucleotides in length. For example, the nucleic acid probe may be at least 200 nucleotides, preferably at least 300 nucleotides, more preferably at least 400 nucleotides, or most preferably at least 500 nucleotides in length. Even longer probes may be used, e.g., nucleic acid probes which are at least 600 nucleotides, at least preferably at least 700 nucleotides, more preferably at least 800 nucleotides, or most preferably at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such organisms may, therefore, be screened for DNA which hybridizes with the probes described above and which encodes a polypeptide having alpha-amylase activity. Genomic or other DNA from such organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA which is homologous with SEQ ID NO: 1 or a subsequence thereof, the carrier material is used in a Southern blot.

For purposes of the present invention, hybridization indicates that the nucleotide sequence hybridizes to a labeled nucleic acid probe corresponding to the nucleotide sequence shown in SEQ ID NO: 1, its complementary strand, or a subsequence thereof, under very low to very high stringency conditions. Molecules to which the nucleic acid probe hybridizes under these conditions can be detected using X-ray film.

For long probes of at least 100 nucleotides in length, very low to very high stringency conditions are defined as prehybridization and hybridization at 42° C. in 5×SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

In a particular embodiment, the wash is conducted using 0.2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency). In another particular embodiment, the wash is conducted using 0.1×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For short probes which are about 15 nucleotides to about 70 nucleotides in length, stringency conditions are defined as prehybridization, hybridization, and washing post-hybridization at about 5° C. to about 10° C. below the calculated Tₘ using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

Under salt-containing hybridization conditions, the effective Tₘ is what controls the degree of identity required between the probe and the filter bound DNA for successful hybridization. The effective Tₘ may be determined using the formula below to determine the degree of identity required for two DNAs to hybridize under various stringency conditions.

Effective Tₘ=81.5+16.6(log M[Na⁺])+0.41(% G+C)−0.72(% formamide)

(See www.ndsu.nodak.edu/instruct/mcclean/plsc731/dna/dna6.htm)

The G+C content of SEQ ID NO: 1 is approximately 55%. For medium stringency, the formamide is 35% and the Na⁺ concentration for 5×SSPE is 0.75 M. Applying this formula to these values, the Effective Tₘ is 76° C.

Another relevant relationship is that a 1% mismatch of two DNAs lowers the Tₘ by 1.4° C. To determine the degree of identity required for two DNAs to hybridize under medium stringency conditions at 42° C., the following formula is used:

% Homology=100−[(Effective Tₘ−Hybridization Temperature)/1.4]

(See www.ndsu.nodak.edu/instruct/mcclean/plsc731/dna/dna6.htm)

Applying this formula to the values, the degree of identity required for two DNAs to hybridize under medium stringency conditions at 42° C. is 100−[(76−42)/1.4]=76%.

In a third aspect, the present invention relates to isolated polypeptides having activity encoded by a polynucleotide comprising nucleotides 97 to 751 of SEQ ID NO: 1, as a unique motif.

The polypeptide may provide the following degradation products of hydrolysis of starch: glucose, maltose, maltotriose, maltotetraose, maltopentose, maltohexose, maltoheptose in addition to dextrins of higher molecular weights. In particular, it may provide the following degradation products of hydrolysis of starch: glucose (DP1), maltose (DP2), maltotriose (DP3), maltotetrose (DP4), maltopentose (DP5), maltohexose (DP6) and maltoheptose (DP7) in approximately similar amounts and a large amount of larger dextrins (>DP10).

The polypeptide may have a molecular weight of approximately 78 kDa. The amylolytic activity may be determined as described below. The alpha-amylase activity may have temperature optimum of approximately 60° C. at pH 6.0, a pH optimum of approximately 6.0 at a temperature of 37° C.

The polypeptide may be an artificial variant with an amino acid sequence derived from the mature part of SEQ ID NO: 2 by substitution (particularly conservative substitution), deletion, and/or insertion of one or more amino acids. Preferably, amino acid changes are of a minor nature, that is conservative amino acid substitutions or insertions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in the parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., alpha-amylase activity) to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309:59-64. The identities of essential amino acids can also be inferred from analysis of identities with polypeptides which are related to a polypeptide according to the invention.

Single or multiple amino acid substitutions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells. Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

The total number of amino acid substitutions, deletions and/or insertions of amino acids 1 to 719 of SEQ ID NO: 2 may be at most 10, at most 9, at most 8, at most 7, at most 6, at most 5, at most 4, at most 3, at most 2, or at most 1.

The polypeptide may have the N-terminal sequence ASGQSLGPVT (positions 1 to 10 in SEQ ID NO: 2).

### Degradation Profiles of the Alpha-Amylase

The degradation products created by degradation of starches depend on the particular alpha-amylase used for the degradation. A typical fungal alpha-amylase such as the Aspergillus oryzae alpha-amylase generates maltose (DP2), maltotriose (DP3) and maltotetrose (DP4) as the main degradation products and leaves a substantial fraction of larger dextrins (>DP10). Bacillus alpha-amylases, such as B. stearothermophilus alpha-amylase and B. licheniformis alpha-amylase, generally produce glucose (DP1), Maltose (DP2), maltotriose (DP3), maltopentose (DP5) or maltohexose (DP6) as the main degradation products and leave only a small fraction of larger dextrins (>DP10).

The alpha-amylase according to the invention produces glucose (DP1), maltose (DP2), maltotriose (DP3), maltotetrose (DP4), maltopentose (DP5), maltohexose (DP6) and maltoheptose (DP7) in approximately similar amounts, evaluated using RI detection, and leaves a substantial amount of larger dextrins (>DP10).

Thus, in a further aspect the invention relates to an alpha-amylase that degrades starch providing glucose (DP1), maltose (DP2), maltotriose (DP3), maltotetrose (DP4), maltopentose (DP5), maltohexose (DP6) and maltoheptose (DP7) in similar amounts and leaves a large amount of larger dextrins (>DP10).

### Sources of Polypeptides Having Alpha-Amylase Activity

A polypeptide of the present invention may be obtained from organisms of any genus. It is preferred that the polypeptide of the present invention is obtained from a microorganism. For purposes of the present invention, the term “obtained from” as used herein in connection with a given source shall mean that the polypeptide encoded by a nucleotide sequence is produced by an organism in which the nucleotide sequence according to the invention has been inserted or is naturally present. In a preferred aspect, the polypeptide obtained from a given source is secreted extracellularly.

### Polynucleotides

The present invention also relates to isolated polynucleotides having a nucleotide sequence which encode a polypeptide of the present invention. In a preferred aspect, the nucleotide sequence is set forth in SEQ ID NO: 1. In another preferred aspect, the nucleotide sequence is the mature polypeptide coding region of SEQ ID NO: 1. The present invention also encompasses nucleotide sequences which encode a polypeptide having the amino acid sequence of SEQ ID NO: 2 or the mature polypeptide thereof, which differ from SEQ ID NO: 1 by virtue of the degeneracy of the genetic code. The present invention also relates to subsequences of SEQ ID NO: 1 which encodes fragments of SEQ ID NO: 2 that have alpha-amylase activity.

The present invention also relates to mutant polynucleotides comprising at least one mutation in the mature polypeptide coding sequence of SEQ ID NO: 1, in which the mutant nucleotide sequence encodes a polypeptide which consists of amino acids 1 to 719 of SEQ ID NO: 2.

The techniques used to isolate or clone a polynucleotide encoding a polypeptide are known in the art and include isolation from genomic DNA, preparation from cDNA, or a combination thereof. The cloning of the polynucleotides of the present invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleotide sequence-based amplification (NASBA) may be used.

In the present invention the polynucleotide having the nucleotide sequence set forth in SEQ ID NO: 1 was isolated using a metagenomic technique i.e. polynucleotides were purified from a soil sample, a polynucleotide library was created from the purified polynucleotides and the library screened to provide the polynucleotide comprising the nucleotide sequence set forth in SEQ ID NO: 2. It is therefore not know which organism the polynucleotide comprising the nucleotide sequence set forth in SEQ ID NO: 1 originates from.

The present invention also relates to polynucleotides having nucleotide sequences encoding polypeptides which have a degree of identity to the mature polypeptide coding sequence of SEQ ID NO: 1 (i.e., nucleotides 97 to 2256) of at least 60%, preferably at least 65%, more preferably at least 70%, more preferably at least 75%, more preferably at least 80%, more preferably at least 85%, more preferably at least 90%, even more preferably at least 95%, and most preferably at least 97% identity, which encode an active polypeptide.

Modification of a nucleotide sequence encoding a polypeptide of the present invention may be necessary for the synthesis of polypeptides substantially similar to the polypeptide. The term “substantially similar” to the polypeptide refers to non-naturally occurring forms of the polypeptide. These polypeptides may differ in some engineered way from the polypeptide isolated from its native source, e.g., artificial variants that differ in specific activity, thermostability, pH optimum, or the like. The variant sequence may be constructed on the basis of the nucleotide sequence presented as the polypeptide encoding region of SEQ ID NO: 1, e.g., a subsequence thereof, and/or by introduction of nucleotide substitutions which do not give rise to another amino acid sequence of the polypeptide encoded by the nucleotide sequence, but which correspond to the codon usage of the host organism intended for production of the enzyme, or by introduction of nucleotide substitutions which may give rise to a different amino acid sequence. For a general description of nucleotide substitution, see, e.g., Ford et al., 1991, Protein Expression and Purification 2: 95-107.

It will be apparent to those skilled in the art that such substitutions can be made outside the regions critical to the function of the molecule and still result in an active polypeptide. Amino acid residues essential to the activity of the polypeptide encoded by an isolated polynucleotide of the invention, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for alpha-amylase activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

The present invention also relates to isolated polynucleotides encoding a polypeptide of the present invention, which hybridize under very low stringency conditions, preferably low stringency conditions, more preferably medium stringency conditions, more preferably medium-high stringency conditions, even more preferably high stringency conditions, and most preferably very high stringency conditions with (i) nucleotides 96 to 2256 of SEQ ID NO: 1, (ii) or (ii) a complementary strand of (i); or allelic variants and subsequences thereof (Sambrook et al., 1989, supra), as defined herein.

The present invention also relates to isolated polynucleotides obtained by (a) hybridizing a population of DNA medium-high, high, or very high stringency conditions with (i) nucleotides 97 to 2256 of SEQ ID NO: 1, or (ii) a complementary strand of (i); and (b) isolating the hybridizing polynucleotide, which encodes a polypeptide having alpha-amylase activity.

### Metagenomic Technique

In the present description and claims the term metagenome is intended to mean a collection of polynucleotides isolated from a sample comprising different organisms.

The sample may be any sample known or suspected to comprise suitable organisms, and is usually collected from a natural environment. The sample may be a sample of soil, water, plant material or other material suspected to comprise potentially interesting polynucleotides.

When the sample has been provided complete genomic material, preferably complete genomic DNA is extracted from the sample using e.g. techniques known within the art such as the method described in (Ausuble et al. 1995 “Current protocols in molecular biology Publ: John Wiley and sons). The obtained genetic material represents organisms that were present in the sample.

When the metagenome has been prepared it is used for creating a metagenomic library which is screened for particular interesting polynucleotide sequences. The creation and screening of the metagenomic library and DNA may be done using methods well known within the area.

One preferred method is preparing a library in an expression vector that subsequently is screened. Several expression vectors have been developed as the skilled person will understand including the lambda ZAP systems, available from Stratagene.

According to the invention may the library be screened using well known screening systems for alpha-amylases, such as plating the library on starch containing agar plates and identifying clones comprising a polynucleotide encoding an alpha-amylase by a clear halo around the clone in question. The starch may be colored in order to facilitate identification of clearing zones around the positive clones or the starch may be dyed after growth of the clones be e.g. placing the plated containing clones of the library in a chamber comprising iodine vapor, whereby the starch will be colored dark and clearing zones will be readily visible.

When positive clones have been identified the isolated polynucleotides may be further characterized and manipulated using well known DNA manipulation techniques such as described in (J. Sambrook, E. F. Fritsch, and T. Maniatis, 1989, Molecular Cloning, A Laboratory Manual, 2d edition, Cold Spring Harbor, N.Y.).

### Nucleic Acid Constructs

The present invention also relates to nucleic acid constructs comprising an isolated polynucleotide of the present invention operably linked to one or more control sequences which direct the expression of the coding sequence in a suitable host cell under conditions compatible with the control sequences.

The skilled person will appreciate that the elements included in a nucleic acid construction suitable for expressing a polypeptide according to the present invention in a suitable host cell depends on the particular selected host cell. In the present invention bacterial cells are the preferred host cells and the nucleic acid constructions according to the invention is therefore described in details for such constructs suitable for use in bacterial cells. However the skilled person will appreciate that other host cells such as fungal cells, mammalian cells, plant cells or insect cells may also be used according to the invention and the skilled person will be capable of deciding which element are suitable for inclusion in nucleic acid constructions intended to use in connection with such host cells.

An isolated polynucleotide encoding a polypeptide of the present invention may be manipulated in a variety of ways to provide for expression of the polypeptide. Manipulation of the polynucleotide's sequence prior to its insertion into a vector may be desirable or necessary depending on the expression vector. The techniques for modifying polynucleotide sequences utilizing recombinant DNA methods are well known in the art.

The control sequence may be an appropriate promoter sequence, a nucleotide sequence which is recognized by a host cell for expression of a polynucleotide encoding a polypeptide of the present invention. The promoter sequence contains transcriptional control sequences which mediate the expression of the polypeptide. The promoter may be any nucleotide sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic alpha-amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (VIIIa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

In a preferred aspect, the promoter is a promoter system consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence, as described in WO 99/43835.

The control sequence may also be a suitable transcription terminator sequence, a sequence recognized by a host cell to terminate transcription. The terminator sequence is operably linked to the 3′ terminus of the nucleotide sequence encoding the polypeptide. Any terminator which is functional in the host cell of choice may be used in the present invention.

The control sequence may also be a suitable leader sequence, a nontranslated region of an mRNA which is important for translation by the host cell. The leader sequence is operably linked to the 5′ terminus of the nucleotide sequence encoding the polypeptide. Any leader sequence that is functional in the host cell of choice may be used in the present invention.

The control sequence may also be a signal peptide coding region that codes for an amino acid sequence linked to the amino terminus of a polypeptide and directs the encoded polypeptide into the cell's secretory pathway. The 5′ end of the coding sequence of the nucleotide sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for bacterial host cells are the signal peptide coding regions obtained from the genes for Bacillus NCIB 11837 maltogenic alpha-amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems.

### Expression Vectors

The present invention also relates to recombinant expression vectors comprising a polynucleotide of the present invention, a promoter, and transcriptional and translational stop signals. The various nucleic acids and control sequences described above may be joined together to produce a recombinant expression vector which may include one or more convenient restriction sites to allow for insertion or substitution of the nucleotide sequence encoding the polypeptide at such sites. Alternatively, a nucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like.

A conditionally essential gene may function as a non-antibiotic selectable marker. Non-limiting examples of bacterial conditionally essential non-antibiotic selectable markers are the dal genes from Bacillus subtilis, Bacillus licheniformis, or other Bacilli, that are only essential when the bacterium is cultivated in the absence of D-alanine. Also the genes encoding enzymes involved in the turnover of UDP-galactose can function as conditionally essential markers in a cell when the cell is grown in the presence of galactose or grown in a medium which gives rise to the presence of galactose. Non-limiting examples of such genes are those from B. subtilis or B. licheniformis encoding UTP-dependent phosphorylase (EC 2.7.7.10), UDP-glucose-dependent uridylyltransferase (EC 2.7.7.12), or UDP-galactose epimerase (EC 5.1.3.2). Also a xylose isomerase gene such as xylA, of Bacilli can be used as selectable markers in cells grown in minimal medium with xylose as sole carbon source. The genes necessary for utilizing gluconate, gntK, and gntP can also be used as selectable markers in cells grown in minimal medium with gluconate as sole carbon source. Other examples of conditionally essential genes are known in the art. Antibiotic selectable markers confer antibiotic resistance to such antibiotics as ampicillin, kanamycin, chloramphenicol, erythromycin, tetracycline, neomycin, hygromycin or methotrexate.

The vectors of the present invention preferably contain an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or nonhomologous recombination. Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication which functions in a cell. The term “origin of replication” or “plasmid replicator” is defined herein as a nucleotide sequence that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

More than one copy of a polynucleotide of the present invention may be inserted into the host cell to increase production of the gene product. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention, which are advantageously used in the recombinant production of the polypeptides. A vector comprising a polynucleotide of the present invention is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be a unicellular microorganism, e.g., a prokaryote, or a non-unicellular microorganism, e.g., a eukaryote.

Useful unicellular microorganisms are bacterial cells such as gram positive bacteria including, but not limited to, a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans and Streptomyces murinus, or gram negative bacteria such as E. coli and Pseudomonas sp. In a preferred aspect, the bacterial host cell is a Bacillus lentus, Bacillus licheniformis, Bacillus stearothermophilus, or Bacillus subtilis cell. In another preferred aspect, the Bacillus cell is an alkalophilic Bacillus.

The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

The host cell may also be a eukaryote, such as a mammalian, insect, plant, or fungal cell.

In a preferred aspect, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a more preferred aspect, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

In an even more preferred aspect, the yeast host cell is a Candida, Hansenula, Kluyveromyces, Pichia, Saccharomyces, Schizosaccharomyces, or Yarrowia cell.

In a most preferred aspect, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. In another most preferred aspect, the yeast host cell is a Kluyveromyces lactis cell. In another most preferred aspect, the yeast host cell is a Yarrowia lipolytica cell.

In another more preferred aspect, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In an even more preferred aspect, the filamentous fungal host cell is an Acremonium, Aspergillus, Aureobasidium, Bjerkandera, Ceriporiopsis, Coprinus, Coriolus, Cryptococcus, Filobasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

In a most preferred aspect, the filamentous fungal host cell is an Aspergillus awamori, Aspergillus fumigatus, Aspergillus foetidus, Aspergillus japonicus, Aspergillus nidulans, Aspergillus niger or Aspergillus oryzae cell. In another most preferred aspect, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another most preferred aspect, the filamentous fungal host cell is a Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, or Ceriporiopsis subvermispora, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

Most preferred is the host cell a bacterial cell such as a Gram positive bacterium belonging to the genus Bacillus.

### Methods of Production

The present invention also relates to methods for producing a polypeptide of the present invention, comprising (a) cultivating a cell, which in its wild-type form is capable of producing the polypeptide, under conditions conducive for production of the polypeptide; and (b) recovering the polypeptide. Preferably, the cell is of the genus Bacillus, and more preferably Bacillus subtilis.

The present invention also relates to methods for producing a polypeptide of the present invention, comprising (a) cultivating a host cell under conditions conducive for production of the polypeptide; and (b) recovering the polypeptide.

The present invention also relates to methods for producing a polypeptide of the present invention, comprising (a) cultivating a host cell under conditions conducive for production of the polypeptide, wherein the host cell comprises a mutant nucleotide sequence having at least one mutation in the mature polypeptide coding region of SEQ ID NO: 1, wherein the mutant nucleotide sequence encodes a polypeptide which consists of amino acids 1 to 719 of SEQ ID NO: 2, and (b) recovering the polypeptide.

In the production methods of the present invention, the cells are cultivated in a nutrient medium suitable for production of the polypeptide using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be recovered using methods known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

### Compositions

The present invention also relates to compositions comprising a polypeptide of the present invention. Preferably, the compositions are enriched in such a polypeptide. The term “enriched” indicates that the alpha-amylase activity of the composition has been increased, e.g., with an enrichment factor of 1.1.

The composition may comprise a polypeptide of the present invention as the major enzymatic component, e.g., a mono-component composition. Alternatively, the composition may comprise multiple enzymatic activities, such as an aminopeptidase, alpha-amylase, carbohydrase, carboxypeptidase, catalase, cellulase, chitinase, cutinase, cyclodextrin glycosyltransferase, deoxyribonuclease, esterase, alpha-galactosidase, beta-galactosidase, gluco-amylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, or xylanase. The additional enzyme(s) may be produced, for example, by a microorganism belonging to the genus Aspergillus, preferably Aspergillus aculeatus, Aspergillus awamori, Aspergillus fumigatus, Aspergillus foetidus, Aspergillus japonicus, Aspergillus nidulans, Aspergillus niger, or Aspergillus oryzae; Fusarium, preferably Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sulphureum, Fusarium toruloseum, Fusarium trichothecioides, or Fusarium venenatum; Humicola, preferably Humicola insolens or Humicola lanuginosa; or Trichoderma, preferably Trichoderma harzianum, Trichoderma koningii, Trichoderma longibrachiatum, Trichoderma reesei, or Trichoderma viride.

The polypeptide compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art.

Examples are given below of preferred uses of the polypeptide compositions of the invention. The dosage of the polypeptide composition of the invention and other conditions under which the composition is used may be determined on the basis of methods known in the art.

### Uses

The alpha-amylase may be used for producing maltodextrins, comprising incubating starch or a starch hydrolysate with the alpha-amylase at suitable conditions so as to hydrolyze alpha-1,4 bonds in the starch or starch hydrolysate. The maltodextrins may be linear or branched, and may include glucose (DP1), maltose (DP2), maltotriose (DP3), maltotetrose (DP4), maltopentose (DP5), maltohexose (DP6) and maltoheptose (DP7).

The maltodextrins may be produced by liquefaction (partial hydrolysis) of starch in the presence of a thermostable alpha-amylase, followed by saccharification (further hydrolysis) of the liquefied starch in the presence of the alpha-amylase of this invention at suitable conditions for cleaving alpha-(1,4) glucosidic bonds.

A very high starch concentration may be processed, e.g. 30% to 40% dry-solids. The liquefaction may include initial hydrolysis for approx. five minutes at approximately 105° C., followed by incubation for approximately one hour at a temperature of 85° to 90° C. to derive a dextrose equivalent (D.E.) of 10 to 15. The saccharification with the alpha-amylase of the invention may be done at pH 5-6.5 and 50-70° C. for 24-72 hours, preferably 36-48 hours. Saccharification with the alpha-amylase of the invention can be done at a higher temperature and a lower pH than saccharification with a conventional fungal amylase.

The alpha-amylase of the invention may also be used for starch conversion, alcohol production, brewing, and baking.

The production of maltose syrup may comprise the steps of: 1) liquefying starch in the presence of an alpha-amylase; 2) dextrinization in the presence of the alpha-amylase of the invention; and 3) recovery of the syrup; and optional purification of the syrup.

The alpha-amylase used for liquefaction in step 1) may be any alpha-amylase. Preferred alpha-amylase are Bacillus alpha-amylases, such as a Termamyl-like alpha-amylase, which including the B. licheniformis alpha-amylase (commercially available as Termamyl™ (Novozymes)), the B. amyloliquefaciens alpha-amylase (sold as BAN (Novozymes), the B. stearothermophilus alpha-amylase (sold as Termamyl™ 120 L type S), The alpha-amylases derived from a strain of the Bacillus sp. NCIB 12289, NCIB 12512, NCIB 12513 or DSM 9375, all of which are described in detail in WO 95/26397, and the alpha-amylase described by Tsukamoto et al., Biochemical and Biophysical Research Communications, 151 (1988), pp. 25-31. Alpha-amylases within the definition of “Termamyl-like alpha-amylase” are defined in for instance WO 96/23874 (Novozymes).

In another aspect the invention relates to a process of producing maltose comprising the steps of: 1) liquefying starch at a temperature of 140-160° C. at a pH of 4-6; 2) dextrinization at a temperature in the range from 60-95° C., in particular at 65-85° C., such as 70-80° C., at a pH 4-6 in the presence of a fungal alpha-amylase variant of the invention; and 3) recovery of the syrup; and optional purification of the syrup.

In an embodiment of the invention an effective amount of gluco-amylase is added in step 2). The syrup will in this embodiment (including treatment with a gluco-amylase) not be maltose syrup, but syrup with a different sugar profile. The gluco-amylase may be an Aspergillus gluco-amylase, in particular an Aspergillus niger gluco-amylase.

Alternatively, the process comprising the steps of: 1) liquefying starch at a temperature of 95-110° C. at a pH of 4-6 in the presence of a Bacillus alpha-amylase; 2) liquefying at a temperature in the range from 70-95° C. at a pH 4-6 in the presence of the alpha-amylase of the invention, followed by recovery and/or optional purification of the product obtained.

Finally, some aspects of the invention relate to various detergent uses. One aspect relates to a detergent additive comprising the alpha-amylase of the invention, optionally in the form of a non-dusting granulate, stabilized liquid or protected enzyme. A preferred embodiment of this aspect relates to a detergent additive which contains 0.02-200 mg of enzyme protein/g of the additive. Another preferred embodiment relates to a detergent additive according to the previous aspect, which additionally comprises another enzyme such as a protease, a lipase, a peroxidase, another amylolytic enzyme and/or a cellulase. Another aspect relates to a detergent composition comprising the alpha-amylase of this invention, and a preferred embodiment of this aspect relates to a detergent composition which additionally comprises another enzyme such as a protease, a lipase, a peroxidase, another amylolytic enzyme and/or a cellulase. Still another aspect relates to a manual or automatic dishwashing detergent composition comprising the alpha-amylase of the invention. A preferred dishwashing detergent composition additionally comprises another enzyme such as a protease, a lipase, a peroxidase, another amylolytic enzyme and/or a cellulase. A final detergent related aspect is a manual or automatic laundry washing composition comprising the alpha-amylase of this invention; and a preferred laundry washing composition according additionally comprises another enzyme such as a protease, a lipase, a peroxidase, an amylolytic enzyme and/or a cellulase.

### Starch Binding Domain

The present invention also relates to nucleic acid constructs comprising a gene encoding a protein operably linked to a nucleotide sequence consisting of a fragment of SEQ ID NO: 1 encoding a polypeptide having starch binding activity, wherein the gene is foreign SEQ ID NO: 1.

The present invention also relates to recombinant expression vectors and recombinant host cells comprising such nucleic acid constructs.

The present invention also relates to methods for producing a protein comprising (a) cultivating such a recombinant host cell under conditions suitable for production of the protein; and (b) recovering the protein.

The nucleotide sequence may be operably linked to foreign genes individually with control sequences as described supra.

The protein may be native or heterologous to a host cell. The term “protein” is not meant herein to refer to a specific length of the encoded product and, therefore, encompasses peptides, oligopeptides, and proteins. The term “protein” also encompasses two or more polypeptides combined to form the encoded product. The proteins also include hybrid polypeptides which comprise a combination of partial or complete polypeptide sequences obtained from at least two different proteins wherein one or more may be heterologous or native to the host cell. Proteins further include naturally occurring allelic and engineered variations of the above mentioned proteins and hybrid proteins.

Preferably, the protein is a hormone or variant thereof, enzyme, receptor or portion thereof, antibody or portion thereof, or reporter. In a more preferred aspect, the protein is an oxidoreductase, transferase, hydrolase, lyase, isomerase, or ligase. In an even more preferred aspect, the protein is an aminopeptidase, alpha-amylase, carbohydrase, carboxypeptidase, catalase, cellulase, chitinase, cutinase, cyclodextrin glycosyltransferase, deoxyribonuclease, esterase, alpha-galactosidase, beta-galactosidase, gluco-amylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase or xylanase.

The gene may be obtained from any prokaryotic, eukaryotic, or other source.

The present invention is further described by the following examples which should not be construed as limiting the scope of the invention.

### Alpha-Amylase Assay

### Dinitrosalicylic Acid Method

For purposes of the present invention, alpha-amylase activity may be determined using the dinitrosalicylic acid method, which is a procedure for the determination of reducing sugar described by Miller, G. L. in (Miller, G. L. 1959. Use of dinitrosalicylic acid for determination of reducing sugar. Anal. Chem. 31:426-428.).

### PNP-G7 Method

Alternatively, alpha-amylase activity may be determined by a method employing PNP-G7 as substrate. PNP-G7 (p-nitrophenyl-alpha,D-maltoheptaoside) is a blocked oligosaccharide which can be cleaved by an endo-amylase. Following the cleavage, the alpha-Glucosidase included in the kit digest the substrate to liberate a free PNP molecule which has a yellow color and thus can be measured by visible spectophotometry at λ=405 nm. (400-420 nm.). Kits containing PNP-G7 substrate and alpha-Glucosidase are available from Boehringer-Mannheim (cat. No. 1054635).

To prepare the substrate one bottle of substrate (BM 1442309) is added to 5 ml buffer (BM1442309). To prepare the α-Glucosidase, one bottle of alpha-Glucosidase (BM 1462309) is added to 45 ml buffer (BM1442309). The working solution is made by mixing 5 ml alpha-Glucosidase solution with 1 ml substrate. The assay is performed by transforming 20 μl enzyme solution to a 96 well microtitre plate and incubating at 25° C. 200 μl working solution, 25° C. is added. The solution is mixed and pre-incubated 1 minute and absorption is measured every 15 sec. over 3 minutes at OD 405 nm. The slope of the time dependent absorption-curve is directly proportional to the specific activity (activity per mg enzyme) of the alpha-amylase in question under the given set of conditions.

### EXAMPLES

Chemicals used as buffers and substrates were commercial products of at least reagent grade.

### Determination of Alpha-Amylase Activity Using the Phadebas Assay

Alpha-amylase activity is determined by a method employing Phadebas® tablets as substrate. Phadebas tablets (Phadebas® Alpha-amylase Test, supplied by Pharmacia Diagnostic) contain a cross-linked insoluble blue-colored starch polymer, which has been mixed with bovine serum albumin and a buffer substance and tabletted.

For every single measurement one tablet is suspended in a tube containing 5 ml 50 mM Britton-Robinson buffer (50 mM acetic acid, 50 mM phosphoric acid, 50 mM boric acid, 0.1 mM CaCl₂, pH adjusted to the value of interest with NaOH). The test is performed in a water bath at the temperature of interest. The alpha-amylase to be tested is diluted in x ml of 50 mM Britton-Robinson buffer. 1 ml of this alpha-amylase solution is added to the 5 ml 50 mM Britton-Robinson buffer. The starch is hydrolyzed by the alpha-amylase giving soluble blue fragments. The absorbance of the resulting blue solution, measured spectrophotometrically at 620 nm, is a function of the alpha-amylase activity.

It is important that the measured 620 nm absorbance after 10 or 15 minutes of incubation (testing time) is in the range of 0.2 to 2.0 absorbance units at 620 nm. In this absorbance range there is linearity between activity and absorbance (Lambert-Beer law). The dilution of the enzyme must therefore be adjusted to fit this criterion. Under a specified set of conditions (temp., pH, reaction time, buffer conditions) 1 mg of a given alpha-amylase will hydrolyze a certain amount of substrate and a blue color will be produced. The color intensity is measured at 620 nm. The measured absorbance is directly proportional to the specific activity (activity/mg of pure alpha-amylase protein) of the alpha-amylase in question under the given set of conditions.

### Media and Solutions

Restriction endonucleases and other enzymes for DNA manipulations were unless otherwise indicated provided from (Supplier) and used according to the instructions of the manufacturer.

### Example 1

### Identification of Sequence SEQ ID No: 1

### A. Genomic Library Construction

A sandy soil sample was taken from a locality in Denmark, Sandbjerg. The soil sample was pasteurized and olive oil enriched. Chromosomal DNA from this enriched culture was prepared by using standard molecular biology techniques (Ausuble et al. 1995 “Current protocols in molecular biology Publ: John Wiley and sons).

The prepared DNA was partially cleaved with the restriction endonuclease Mbol and separated in a sucrose gradient by ultracentrifugation. Fragments of 3 to 10 kilobases were extracted, precipitated and resuspended in a suitable buffer.

A genomic library was made by using the Stratagene ZAP Express™ predigested Vector kit and Stratagene ZAP Express™ predigested Gigapack® cloning kit (BamH I predigested) (Stratagene Inc., USA) following the instructions/recommendations from the vendor.

The resulting lambdaZAP library comprised of 200 pfu/ul of which 100,000 pfu were collected for mass excision. The resulting 41,900,000 cfu/ul E. coli colonies were pooled and plasmids were prepared by using the Qiagen Spin Mini prep kit (Qiagen, Germany).

### B. Library Screening

Approximately 100.000 library clones were evenly spread on plates containing Cibachron red stained amylopectin (1% (w/w)), LB agar and kanamycin (25 micro-g/ml).

After incubation over night at 37° C. positive clones were identified by a clearing zone around the colony indicating excretion of a starch degrading enzyme.

One positive clone (pSBL0622-2) of this library was identified. The extracellular production of a starch degrading enzyme by this clone was verified by growing the clone over night on LB plates containing kanamycin (25 micro-g/ml) and 1% (w/w) potato starch (Sigma, S-2630), followed by iodine vapor staining of the starch, which indicated clearing zone around the colonies where the starch was degraded. In addition, a band of the calculated molecular weight (78 kDa), corresponding to the calculated weight of the mature peptide from SEQ ID NO: 2 position 1-719, on a SDS-Page gel indicated expression of the enzyme. The screening host (E. coli DH10B) alone did not show this protein band.

The plasmid of the active clone was prepared by using the Qiagen Plasmid Preparation Kit according to the manufacturer's recommendations.

### C. Analysis of the Gene

The gene sequence from that plasmid was obtained by Sanger sequencing using the T3 and T7 sequencing primers having annealing sites in the vector, and custom made sequencing primers were designed based on the obtained sequences as shown in SEQ ID NO: 3-8.

The complete DNA sequence encoding the alpha-amylase of the invention is shown as SEQ ID NO: 1, and the deduced amino acid sequence is shown as SEQ ID NO: 2, and the signal peptide is identified.

The deduced amino acid sequence was analyzed, and the full length polypeptide showed a domain structure including
an alpha-amylase protein sequence which can be classified as a member of the glycosyl hydrolase family 13 subfamily 2 (Mark R. Stam et al., “Dividing the large glycoside hydrolase family 13 into subfamilies: towards improved functional annotations of a-alpha-amylase-related proteins”, Protein Engineering, Design & Selection vol. 19 no. 12 pp. 555-562, 2006) and comprises a N-terminal signal peptide sequence, the GH13_{—}2 domain,
an alpha-amylase C-terminal domain (Pfam acc. No. PF02806) and
a carbohydrate binding domain (CBM) 20 (Pfam PF00686).

### Example 2

### Cloning of Alpha-Amylase Gene in Bacillus subtilis

The signal peptide from the alpha-amylase from B. licheniformis (AmyL) was fused by PCR in frame to the gene encoding the alpha-amylase. The DNA coding for the resulting coding sequence was integrated by homologous recombination on the Bacillus subtilis host cell genome. The gene construct was expressed under the control of a triple promoter system (as described in WO 99/43835), consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence. The gene coding for Chloramphenicol acetyl-transferase was used as maker. (Described e.g. in Diderichsen et al., A useful cloning vector for Bacillus subtilis. Plasmid, 30, p. 312, 1993).

Ten Chloramphenicol resistant transformants were selected, grown in 4 mL cultures in soy based media for 3 days at 37° C. and 20 micro-L of the supernatants were spotted on agar plates containing cibachron-red labeled amylopectin. One such clone was selected analyzed by DNA sequencing to verify the correct DNA sequence of the construct.

Fermentations of the alpha-amylase expression clone was performed on a rotary shaking table in 300 mL baffled Erlenmeyer flasks each containing 100 mL soy based media supplemented with 34 mg/l chloramphenicol. The clone was fermented for 4 days at 37° C.

The enzyme was purified from the supernatant using chromatography on a Phenyl Sepharose column and then on a Q Sepharose column eluted using a salt gradient. The pool had absorption values of A280=0.79 and A260=0.43 and the purified amylase was the major band on an 12% SDS-PAGE gel. The purified enzyme was used in Examples 4, 5 and 6.

### Example 3

### Detection of Alpha-Amylase Activity

A. Assay with Dinitrosalicylic Acid.

Determination of amylolytic activity was carried out by using the dinitrosalicylic acid method, which is a procedure for the determination of reducing sugar (Miller, G. L. 1959. Use of dinitrosalicylic acid for determination of reducing sugar. Anal. Chem. 31:426-428.).

B. Detection with a Zymogram:

Two hundred micro liter of culture supernatant from expression clones (Example 2) carrying the alpha-amylase gene (SEQ ID No: 1) were precipitated by the addition of trichloroacetic acid to a final concentration of 10% following an incubation for 30 min in an ice-bath and centrifugation for 5 min at 13,000×g. The supernatant was removed and the samples (50 μL) were size excluded on a 10% SDS-PAGE gel. The gel was washed in 2.5% Triton X-100 containing 100 mM Tris pH 8 and 5 mM CaCl₂ for 30 min at RT following an incubation in 1% Paselli starch (Avebe, Netherlands), 100 mM Tris pH 8 and 5 mM CaCl₂ for 30 min at room temperature. The gel was stained in with Lugol solution (0.15 g I₂, 1.5 g KI dissolved in 100 mL water) for 10 seconds and remaining Lugol solution was washed away with excess water.

The SDS-PAGE and zymogram of expression clones carrying the alpha-amylase gene (SEQ ID NO: 2) indicated the protein band and clearing zone of the expected size (78 kDa).

### Example 4

### N-Terminal

The N-terminal sequence was determined to be ASGQSLGPVT (positions 1 to 10 in SEQ ID NO: 2).

### Example 5

### pH and Temperature Optima

Trials regarding pH optimum were carried out by running Phadebas assay at 37° C. in a buffer adjusted to different pH values buffer from pH 2.0 to pH 9.0. The pH optimum of the enzyme was found to be approximately pH 6.

Similarly, trials regarding temperature optimum were carried out by running Phadebas assay at pH 6 at temperature ranging from 30° C. to 80° C. The temperature optimum of the enzyme was found to be determined approximately 60° C.

### Example 6

### Starch Degradation Profile

The starch degradation profile was carried out on 5% (w/w) waxy corn starch (Cerestar waxy maize 0401) prepared in 50 mM acetate buffer, 1 mM CaCl₂, pH 6.0 (boiled 3 minutes to solubilize the starch). Purified enzyme as prepared in Example 2 was added to 1 ml substrate in an amount of 0.2 mg enzyme/g dry matter, and the mixture was incubated for 24 hours at 50° C. One drop concentrated HCl was added and the sample was incubated 15 minutes at 100° C. to inactivate the enzyme. The sample was filtered through a 0.22 micro-m filter and analyzed on a Waters HPLC system using a BIO-RAD Aminex® HPX-42A column (Cat. nr. 125-0097), a BIO-RAD Deashing Holder (Cat. nr. 125-0139) fitted with 1 set of BIO-RAD Micro-Guard Deashing cartridges (Cat. no. 125-0118), eluted with double degassed distilled water and detected using a Waters RI detector.

The degradation profile showed glucose (DP1), maltose (DP2), maltotriose (DP3), maltotetrose (DP4), maltopentose (DP5), maltohexose (DP6) and maltoheptose (DP7) in approximately similar amounts, evaluated using RI detection, and left a substantial amount of larger dextrins (>DP10).

1-11. (canceled)

12. An isolated polypeptide having alpha-amylase activity, selected from the group consisting of:
(a) a polypeptide comprising an amino acid sequence which has at least 65% identity with the sequence of amino acids 1 to 719 of SEQ ID NO: 2;
(b) a polypeptide encoded by a polynucleotide which hybridizes under low stringency conditions with (i) nucleotides 97 to 2256 of SEQ ID NO: 1, or (ii) a complementary strand of (i); and
(c) a fragment of the sequence of amino acids 1 to 719 of SEQ ID NO: 2 which has alpha-amylase activity.

13. The polypeptide of claim 12, which comprises an amino acid sequence which has at least 65% identity with amino acids 1 to 719 of SEQ ID NO: 2.

14. The polypeptide of claim 12, which comprises an amino acid sequence which has at least 70% identity with amino acids 1 to 719 of SEQ ID NO: 2.

15. The polypeptide of claim 12, which comprises an amino acid sequence which has at least 75% identity with amino acids 1 to 719 of SEQ ID NO: 2.

16. The polypeptide of claim 12, which comprises an amino acid sequence which has at least 80% identity with amino acids 1 to 719 of SEQ ID NO: 2.

17. The polypeptide of claim 12, which comprises an amino acid sequence which has at least 85% identity with amino acids 1 to 719 of SEQ ID NO: 2.

18. The polypeptide of claim 12, which comprises an amino acid sequence which has at least 90% identity with amino acids 1 to 719 of SEQ ID NO: 2.

19. The polypeptide of claim 12, which comprises an amino acid sequence which has at least 95% identity with amino acids 1 to 719 of SEQ ID NO: 2.

20. The polypeptide of claim 12, which comprises the sequence of amino acids 1 to 719 of SEQ ID NO: 2.

21. The polypeptide of claim 12, which consists of the sequence of amino acids 1 to 719 of SEQ ID NO: 2.

22. A process for producing maltodextrins, comprising incubating starch or a starch hydrolysate with the polypeptide of claim 12 so as to hydrolyze alpha-1,4 bonds in the starch or starch hydrolysate.

23. An isolated polynucleotide comprising a nucleotide sequence which encodes a polypeptide of claim 12.

24. A nucleic acid construct comprising a polynucleotide of claim 23 operably linked to one or more control sequences that direct the production of the polypeptide in an expression host.

25. A recombinant expression vector comprising the nucleic acid construct of claim 24.

26. A recombinant host cell comprising the nucleic acid construct of claim 24.

27. A method for producing a polypeptide having alpha-amylase activity, comprising
(a) cultivating the recombinant host cell of claim 26 under conditions conducive for production of the polypeptide; and
(b) recovering the polypeptide.
